(19) United States
(12) Patent Application Publication
(10) Pub. No.: US 2025/0278886 A1
(43) Pub. Date: Sep. 4, 2025

(54) Computed Tomography Based Textured Surfaces

(71) Applicant: Lumafield, Inc., Cambridge, MA (US)

(72) Inventors: William Jacob Hartman, Northfield, VT (US); Daniel Micah Pipe-Mazo, Redondo Beach, CA (US); Sahil Rajesh Shah, Cambridge, MA (US); Sebastian Gerd Ahling, Medford, MA (US)

(21) Appl. No.: 18/990,120

(22) Filed: Dec. 20, 2024

Related U.S. Application Data
(60) Provisional application No. 63/559,792, filed on Feb. 29, 2024.

(51) Int. Cl. G06T 15/04; A61B 6/00; A61B 6/03; G06T 7/10; G06T 15/20; G16H 30/20; G16H 30/40

(52) U.S. Cl. CPC G06T 15/04; A61B 6/032; A61B 6/5241; G06T 7/10; G06T 15/20; G16H 30/20; G16H 30/40; G06T 2207/10081

(57) ABSTRACT

Provided herein are methods, apparatuses, computer program products, and systems for generating textured surfaces using computed tomography. One method can include generating a computer data structure representing three-dimensional surfaces of a physical object from (i) segmentation of volumetric data reconstructed from radiographs, (ii) position information and orientation information of the physical object, and (iii) size information of the physical object; adding texture data to the three-dimensional surfaces represented in the computer data structure using images captured by at least one optical camera, the images being of the physical object at different rotational orientations, by projecting data of the images onto the three-dimensional surfaces using the size information, the position information, and the orientation information of the physical object and predetermined position, location, and parameters of the at least one optical camera; and providing the computer data structure for processing of the three-dimensional surfaces.

### CROSS-REFERENCE TO RELATED APPLICATIONS

This patent application claims priority to and the benefit of U.S. Provisional Patent Application No. 63/559,792, filed on Feb. 29, 2024, which is incorporated herein by reference in its entirety.

### BACKGROUND

Three-dimensional (3D) X-ray computed tomography (CT) is a powerful inspection technology for both individual parts as well as assembled final products. 3D scan data from a CT scan combines a series of X-ray projections taken from different projection angles and uses computer processing techniques to create a 3D reconstruction of the scan object. 3D X-ray CT can be used to make quality decisions about a part or to monitor upstream processes.

Textured surfaces of a physical object include data representing 3D surfaces of the physical object and texture data of the 3D surfaces. Textured surfaces can be used for quality control and/or inspection of a physical object. Some existing techniques that generate textured surfaces use optical cameras, such as standard RGB cameras and depth-based cameras, to produce both surface scans and surface texture data. Some existing techniques that generate textured surfaces use photogrammetry, gaussian splatting, or other complex camera techniques to produce both the 3D surfaces and the texture data. However, these existing techniques cannot generate accurate surface representations, especially when objects being imaged have visual occlusions, such as overhangs or other complex structures.

### SUMMARY

This specification describes technologies relating to generating a computer data structure representing both 3D surfaces of a physical object and texture data of the surface of the physical object using a multimodal X-ray system that includes a CT scanner and one or more cameras. The specification also describes technologies relating to the design and usage of a reference object (referred to as a “phantom”) to calibrate location and orientation of the one or more cameras relative to the CT scanner.

Particular embodiments of the subject matter described in this specification can be implemented to realize one or more of the following advantages. The systems and techniques can produce a computer data structure of an object that includes both a surface representation, e.g., an accurate 3D surface mesh, of the object, and an accurate texture, e.g., color data, for the surface of the object. The systems and techniques described in this specification uses X-ray CT to obtain data representing surfaces of a physical object and can produce a definitive, accurate surface representation for complex objects for which some existing techniques struggle. The systems and techniques use one or more optical cameras to add texture data to the surface representation, producing superior surface accuracy on objects in which some existing techniques might fail, and comparable surface texturing information. The systems and techniques described in this specification can generate accurate 3D surfaces from volumetric data reconstructed from a CT scan, and can add texture data to the 3D surfaces using images captured from at least one optical camera based on the predetermined parameters of the system, without using photogrammetry, gaussian splatting, or other camera techniques which can be more complex and need to produce both the 3D surfaces and the texture data.

Some existing techniques, such as photogrammetry, might produce inaccurate surface representation for some surface material, sheen, texture, and composition due to the inference limitations of using only visible light data generated by the cameras. Some optical camera imaging techniques using visible light, infrared light, or laser scanning cannot produce accurate surfaces for objects with transparent surfaces, shiny/reflecting surfaces, furry surfaces, overlapping surfaces, or other surfaces. The systems and techniques described in the specification can produce accurate surfaces regardless of surface material, sheen, texture, and composition because the surface representation is generated from volumetric data reconstructed from radiographs acquired using a CT scanner.

Some existing techniques cannot achieve accurate calibration of location and orientation of cameras relative to a CT scanner. Failure to achieve accurate calibration can result in misalignment of the projected image features with the X-ray reconstruction geometry, e.g., producing a loss of details and blurring in textured surfaces. For example, small angular errors in the calibration can cause large translation shifts (abbe errors) at a surface of a scan object. Some existing techniques, such as photogrammetry, can locate the camera positions relative to an X-ray scanner based on alignment of meshes of a target object. Photogrammetry techniques can generate a mesh of the target object from several optical images taken from different orientations, and can align the mesh with another mesh generated from an X-ray reconstruction volume to locate the camera position relative to the X-ray scanner. However, photogrammetry techniques are computationally intensive and require more images than performing camera calibration using optically detectable features, and additional images and computations are required and repeated for each camera of a photogrammetry system.

The systems and techniques described in this application include performing accurate multimodal calibration of location and orientation of one or more cameras relative to a CT scanner using a phantom. The phantom includes both X-ray detectable features and optically detectable features at fixed known locations relative to each other. Based on the result of the calibration, the present systems and techniques can accurately align images captured by the one or more cameras with a CT reconstruction volume obtained by the CT scanner. By using a phantom that includes both X-ray detectable features and optically detectable features, the present systems and techniques can minimize the number of different phantoms required for the calibration. The present systems and techniques can eliminate variation introduced from movement of a sample stage of the multimodal X-ray system for adapting different phantoms or changes in weights of the different phantoms. Rather than using computationally intensive photogrammetry techniques that require taking additional images of the phantom for each camera and performing calibration computations for each camera, the present systems and techniques can calibrate multiple cameras located in any position within an enclosure of the multimodal X-ray system using a single set of radiographs and optical images of the phantom. Thus, adding more cameras to the multimodal X-ray system does not add more calibration steps.

The details of one or more embodiments of the subject matter described in this specification are set forth in the accompanying drawings and the description below. Other features, aspects, and advantages of the invention will become apparent from the description, the drawings, and the claims.

### BRIEF DESCRIPTION OF THE DRAWINGS

FIG. 1 shows an example system that generates textured surfaces.

FIG. 2 is a flowchart showing an example of a process to generate textured surfaces.

FIG. 3 is a flowchart showing an example of a process to generate textured surfaces in UV coordinates.

FIGS. 4A-4C shows an example of a reference object.

FIG. 5 is a flowchart showing an example of a process of multimodal calibration using a reference object.

FIG. 6 is a flowchart showing an example of a process to determine an object boundary in a reconstruction volume using a camera image.

FIG. 7 is a flowchart showing an example of a process to estimate part occupation.

Like reference numbers and designations in the various drawings indicate like elements.

### DETAILED DESCRIPTION

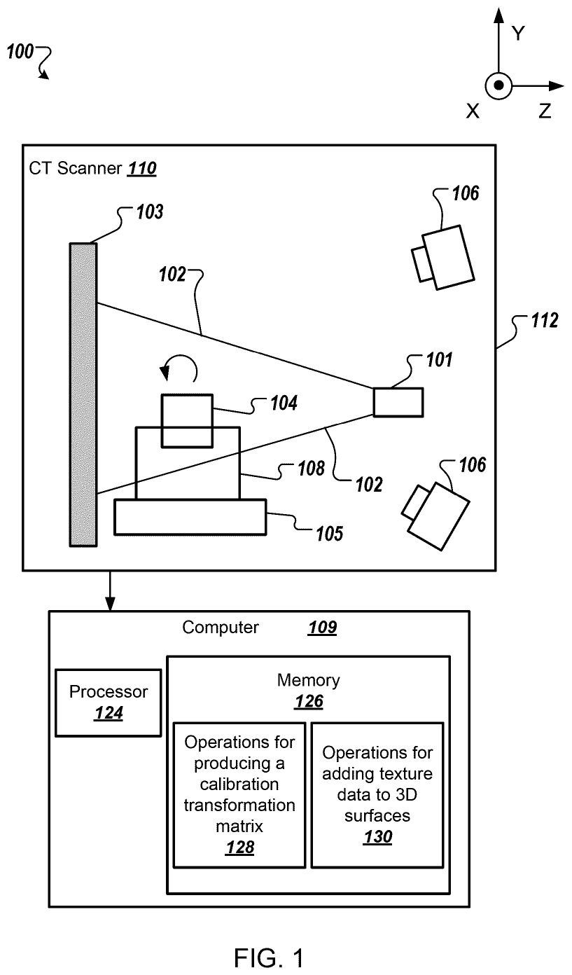

FIG. 1 shows an example system 100 that generates textured surfaces. The system 100 includes a computed tomography (CT) scanner 110. The CT scanner 110 includes an X-ray source 101 configured to emit X-rays 102. The CT scanner 110 includes a detector 103 arranged to receive the X-rays 102 after interaction with an object 104 that has been placed in the CT scanner 110. The object 104 is located between the X-ray source 101 and the detector 103. The system 100 includes at least one optical camera 106. The at least one optical camera 106 can capture one or more optical images of the object 104. In some implementations, the object 104 can be a physical object and the system 100 can perform a CT scan of the physical object. In some implementations, the object 104 can be a reference object (referred to as a “phantom”) used to calibrate location and orientation of the at least one optical camera 106 relative to the CT scanner 110.

The system 100 can take a CT scan of a physical object placed in the CT scanner 110. The physical object can be proximate to a location within the CT scanner. For example, the object 104 can be placed on a turntable 105 and the object 104 can be proximate to the location of the turntable in the CT scanner 110. In some implementations, the system 100 can include a fixture 108 located on top of the turntable 105 that holds the object 104 during a scan. In some implementations, the fixture 108 can be made of an X-ray transmissive material. For example, the fixture 108 can be made of closed-cell extruded polystyrene foam. When the turntable 105 rotates, the fixture 108 and the object 104 rotate at the same speed and in the same rotational direction as the turntable 105.

The system 100 can acquire two-dimensional (2D) radiographs of the physical object. The CT scan of the object can include 2D radiographs of the object at different rotational orientations. For example, the object 104 can be placed on the turntable 105 and the CT scan of the object can include 2D radiographs of the object while the turntable rotates to different rotational positions of the turntable.

The system 100 can reconstruct volumetric data for the physical object. The system can generate volumetric data reconstructed from the 2D radiographs of the physical object. The volumetric data can represent the density of the object. For example, each voxel in the volumetric data can have a value representing the density of the object at the location of the voxel.

The system 100 can determine, from the 2D radiographs, position information and orientation information of the physical object with respect to the known location within the CT scanner 110, e.g., the location of the turntable 105. During the CT scan, the system 100 can track the position of the turntable on which the object is located. The system 100 can determine the location of the center of the object within the CT scanner and within the field of view (FOV) of the CT scanner 110 using the 2D radiographs and the position of the turntable. A FOV is the maximum volume of a scan object that can be captured in an X-ray CT scan, defining the visible region within the X-ray CT scanner. An FOV can be either larger or smaller than the scan object. When the FOV is larger than the scan object, the entire scan object is imaged. When the FOV is smaller than the scan object, only part of the scan object is imaged.

The system can determine the position and tilt angle of the object 104 relative to the center of the turntable 105. Thus, the system can precisely determine the location of the volumetric data in real-world 3D space. When performing a CT reconstruction from the 2D radiographs, the system can run one or more CT reconstruction algorithms on the 2D radiographs to determine the axis of rotation of the object. Based on the axis of rotation of the object, the system can determine the position and orientation of the object in real-world 3D space.

The system 100 can determine size information of the physical object from a predetermined distance between the known location, e.g., the center of the turntable 105, and the X-ray source 101. The precise position of the center of the turntable 105 is known and calibrated with respect to the X-ray source 101, e.g., during manufacturing of the CT scanner 110. The cone formed by the X-ray 102 represents the cone beam of the X-ray source 101. The system can determine the real-world size of the object 104 based on the position of the turntable 105 in the Z axis relative to the X-ray source 101 and the detector 103.

The system 100 can segment the volumetric data. The system 100 can generate a computer data structure representing 3D surfaces of the physical object from (i) segmentation of the volumetric data reconstructed from 2D radiographs of the object, (ii) position information and orientation information of the physical object, and (iii) size information of the physical object. In some implementations, the system can run a segmentation analysis on the volumetric data to extract a 3D surface mesh. The 3D surface mesh can include vertices and edges that define polygons that approximate the surface of the physical object as determined by the volumetric data.

For example, using the position of the turntable, the object's axis of rotation, and the known real-world size of the voxels in the volumetric data, the system can determine the real-world coordinates of the 3D surface mesh. In some implementations, the segmentation of the volumetric data can be performed using a global threshold derived via ISO-50 method. In the ISO-50 method, the gray value that is equidistant (e.g., 50% from) the background and material gray value peaks is used as the global threshold for a segmentation operation. The system can use this threshold value as an input to a surface meshing algorithm, such as marching cubes, which interpolates local voxel gray values at the threshold value to produce a surface mesh with sub-voxel spatial resolution. In some implementations, other segmentation techniques (e.g., random forest, or one or more deep neural networks, such as U-Net, Mask Region-based Convolutional Neural Network (R-CNN)) can be used to segment the volumetric data. In some implementations, other suitable meshing techniques can be used to extract a triangular or quadrilateral mesh, or another suitable type of mesh.

The system 100 includes at least one optical camera 106. The at least one optical camera 106 captures images of the physical object at different rotational orientations. For example, the system 100 can include one or more visible light cameras inside the enclosure 112 of the CT scanner 110. In some implementations, the at least one optical camera 106 can include a non-visible light camera, such as an infrared camera, an ultraviolet (UV) camera, or a thermal camera. The at least one optical camera 106 can be an RGB camera or mono-color camera, a fixed focus camera or a variable focus camera, a fixed aperture camera or a variable aperture camera, a polarized camera or an unpolarized camera, or any suitable optical camera. The at least one optical camera can be mounted at a known real-world position inside of the CT scanner at a predetermined orientation (e.g., facing direction of the camera), with predetermined parameters of the camera, such as predetermined camera intrinsic and lensing parameters.

The system 100 obtains images of the physical object 104 captured using the at least one optical camera 106. In some implementations, the system 100 can capture the images of the physical object 104. In some implementations, an end user of the system can perform the capturing of the images and can provide the images to the system 100. In some implementations, images of the physical object 104 can be captured using the at least one optical camera 106 during a CT scan, e.g., while acquiring the 2D radiographs that are used to generate the volumetric data of the physical object 104. That is, acquiring the 2D radiographs and capturing the images can be performed concurrently, e.g., during the same rotation of the physical object. In some implementations, acquiring the 2D radiographs and capturing the images can be performed sequentially, e.g., in separate rotations of the physical object. In some implementations, the system 100 can include additional lighting to the interior of the CT scanner 110 to improve the quality of the images captured by the at least one optical camera 106. In some implementations, the system can include a white or other colored background on the interior of the CT scanner that is within the FOV of the camera(s) to improve the quality of the images. The system can use other suitable techniques to improve the quality of the images.

The system 100 can obtain a corresponding rotation orientation for each image captured by the at least one camera. For example, the images of the physical object 104 can be captured using the at least one optical camera 106 while the turntable rotates through 360 degrees. For each image, the system can obtain a corresponding rotation angle of the turntable 105. The captured images can be as few as tens of images or as many as thousands of images across a predetermined rotation range, e.g., 360 degrees rotation, of the object. The images can be captured at evenly spaced rotational orientations, or unevenly spaced rotational orientations. For example, the system can capture N images at 360/N degrees apart.

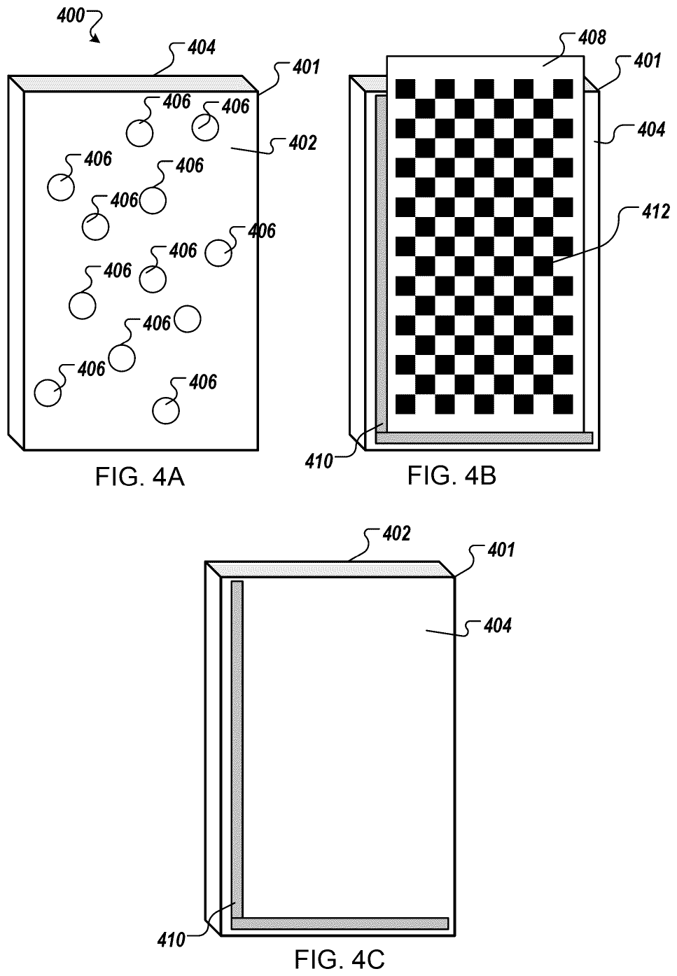

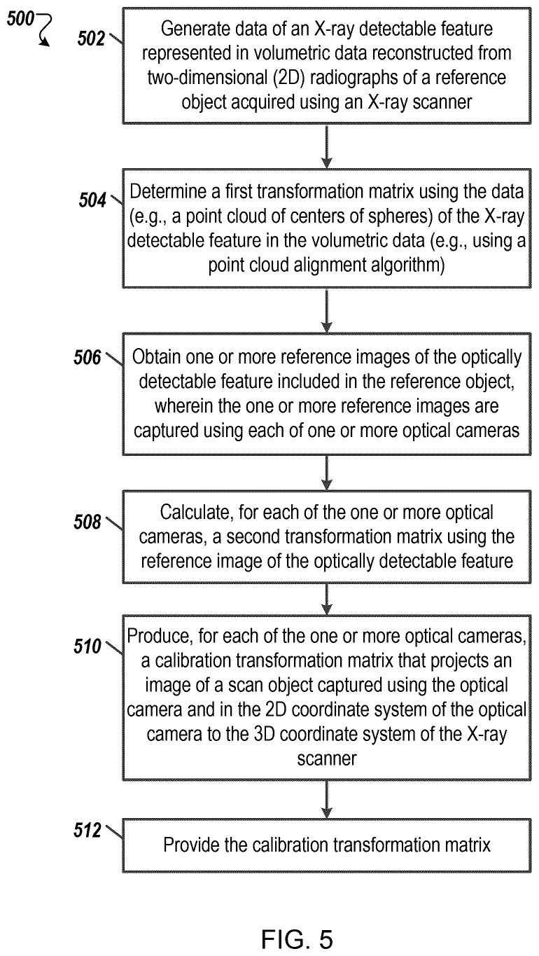

The system 100 can add texture data to the 3D surfaces represented in the computer data structure using the images captured by the at least one optical camera. For example, the system can generate texture data for a 3D surface mesh using the images and their respective predetermined rotational positions of the turntable. The system 100 can project data of the images onto the 3D surfaces using the size information, position information, and orientation information of the physical object and the predetermined position, location, and parameters of the at least one optical camera. In some implementations, the system 100 can project the data of the images onto the 3D surfaces using a calibration transformation matrix. Details of generating the calibration transformation matrix are described herein in connection with FIGS. 4A-4C and FIG. 5.

The system 100 can provide the computer data structure for processing of the 3D surfaces. In some implementations, the system can provide the computer data structure for quality control and/or inspection of a physical object. For example, during production, a quality control or inspection procedure can perform go/no-go checks on products using textured surface data of the products to ensure that the products are of high quality.

In some implementations, the computer data structure can be digital twins of physical objects. In some implementations, the system can provide the computer data structure for renderings and/or simulations of scenes, facilities, or other processes that use digital twins of physical objects. For example, a computer aided design program or a computer aided simulation program can perform analysis using the texture data and the 3D surfaces of the physical object.

In some implementations, the system can provide the computer data structure as digital copies of physical objects. For physical objects that are of high value, such as fine art, archeological artifacts, or high-valued objects in other applications, examining the original physical object can be undesirable or impractical for security or safety reasons. Also, it can be important to preserve accurate digital copies of the high-valued objects. The systems and techniques described in the specification can create an archive of accurate digital copies of physical objects that are of high value, allowing convenient examination of the digital copies of the physical objects.

In some implementations, the system can provide the computer data structure for processing of the textured surface data to determine an identifier on the surface of the physical object. For example, the identifier can be a unique identifier assigned for a physical object, and can be an alphanumeric code or a data matrix code written, printed, or engraved on the surface of the physical object. The system can use the identifier for inventory tracking of the physical object. After determining the identifier using the textured surface data of an object, the system can associate a scan of an object with the unique object corresponding to the identifier.

In some implementations, the system can provide the computer data structure for generating training examples that can be used to train a machine learning model, e.g., a generative artificial intelligence (AI) model or a deep neural network. 2D or 3D training examples of one or more physical objects at different perspectives, under different lighting conditions, or in different scenes can be generated using the textured surface data of the one or more physical objects.

The system 100 includes a computer 109. The computer 109 can be one or more computers that are integrated with the CT scanner 110, and/or located remotely from the CT scanner 110 (e.g., at a remote server and communicatively coupled with the CT scanner 110, e.g., over the Internet). The computer 109 can include at least one processor 124. The processor(s) 124 can be embodied by any computational or data processing device, such as a central processing unit (CPU), application specific integrated circuit (ASIC), or comparable device. The processor(s) 124 can be implemented as a single controller, or a plurality of controllers or processors.

The computer 109 can include at least one memory 126. The memory 126 can be fixed or removable. The memory 126 can encode computer program instructions or computer code contained therein. Memory 126 can be any suitable storage device, such as a non-transitory computer-readable medium. The term “non-transitory,” as used herein, can correspond to a limitation of the medium itself (i.e., tangible, not a signal) as opposed to a limitation on data storage persistency (e.g., random access memory (RAM) vs. read-only memory (ROM)). A hard disk drive (HDD), random access memory (RAM), flash memory, or other suitable memory can be used. The one or more memories can be combined on a same integrated circuit as one or more processors, or can be separate from the one or more processors. Furthermore, the computer program instructions stored in the memory, and which can be run by the processor(s), can be any suitable form of computer program code, for example, a compiled or interpreted computer program written in any suitable programming language. In some implementations, the computer 109 can store computer data structure representing the 3D surfaces of a physical object, the texture data, 2D radiographs acquired by the CT scanner 110, volumetric data reconstructed from the 2D radiographs, or a combination of these, in the memory 126.

The processor 124, the memory 126, and any subset thereof, can be configured to perform one or more processes including CT scanning and data acquisition, post-processing, reconstructing the volumetric data, segmenting the volumetric data, capturing the images using the at least one optical camera, producing a calibration transformation matrix, projecting the images onto the 3D surfaces, and adding texture data to the 3D surfaces represented in the computer data structure. For example, the memory 126 can store instructions configured to cause the computer 109 to perform operations including operations 128 for producing a calibration transformation matrix, operations 130 for adding texture data to 3D surfaces, or both. The memory and the computer program instructions can be configured, with the processor for the particular device, to cause a hardware apparatus to perform one or more of the processes described in this application. Therefore, in some implementations, a non-transitory computer-readable medium is encoded with computer instructions that, when executed in hardware, perform a process such as one of the processes described herein. In some cases, one or more of the processes described herein are implemented entirely in hardware.

As noted above, in some implementations, these processes can be performed by the CT scanner 110, and so no separate computer is needed. In such implementations, the computer 109 and the CT scanner 110 are integrated into a single device, rather than being in separate devices as shown in FIG. 1. In some implementations, the CT scanner 110 is an inexpensive scanning device with minimal processing capabilities, and a separate computer is communicatively coupled with the CT scanner 110 and is configured to perform one or more of the processes described herein.

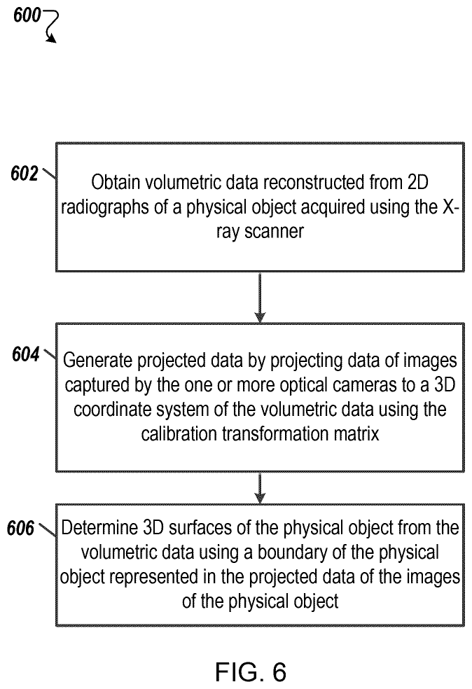

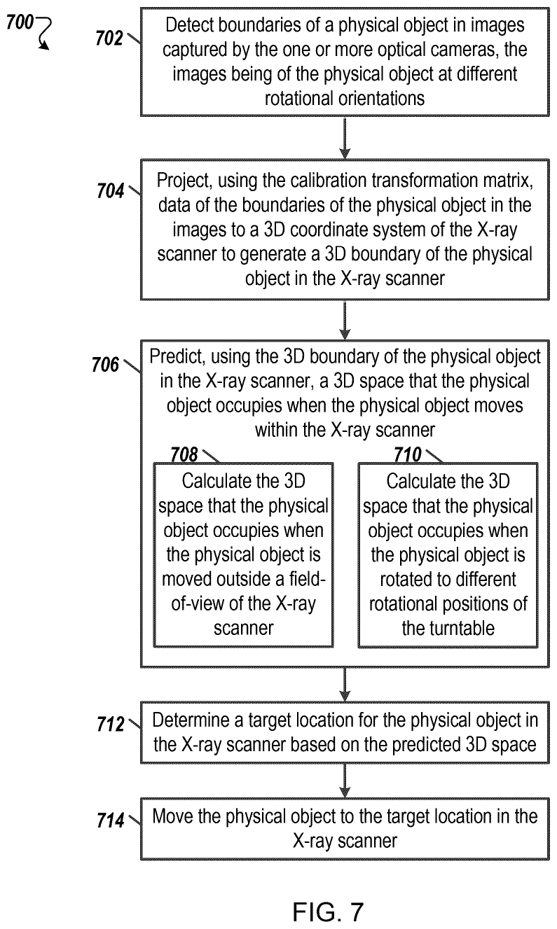

In some implementations, the system 100 can be configured to determine a calibration transformation matrix that can be used to calibrate location and orientation of the one or more optical cameras 106 relative to the CT scanner 110. Details of generating the calibration transformation matrix are described herein in connection with FIGS. 4A-4C and FIG. 5. In some implementations, the system 100 can be configured to determine an object boundary in a reconstruction volume using a camera image. Details of determining an object boundary in a reconstruction volume using a camera image are described herein in connection with FIG. 6. In some implementations, the system 100 can be configured to estimate part occupation. Details of estimating part occupation are described herein in connection with FIG. 7.

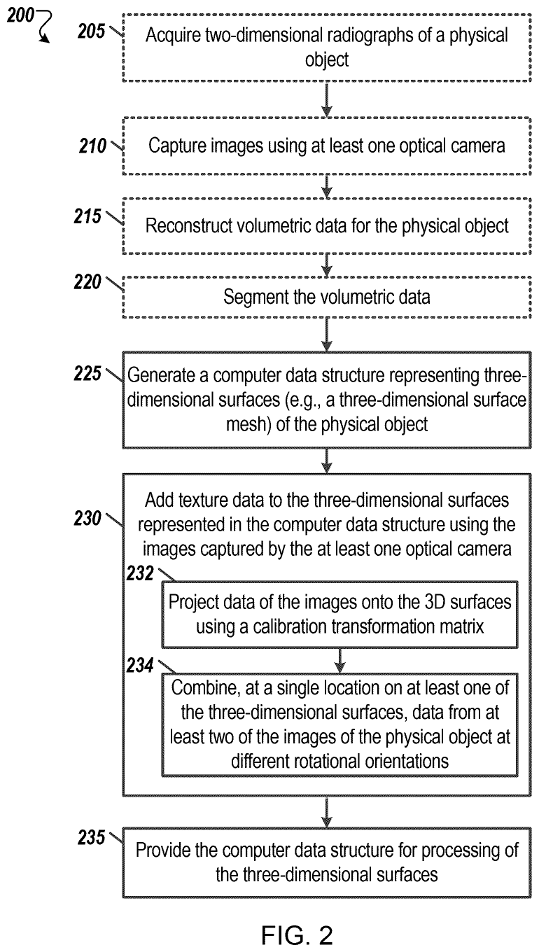

FIG. 2 is a flowchart showing an example of a process 200 to generate textured surfaces. The process 200 can be performed by a system of one or more computers located in one or more locations. For example, a multimodal X-ray system, e.g., the system 100 depicted in FIG. 1, appropriately programmed in accordance with this specification, can perform the process 200.

The system generates (225) a computer data structure representing 3D surfaces of a physical object from (i) segmentation of volumetric data reconstructed from 2D radiographs of the physical object acquired using an X-ray source in a CT scanner when the physical object is proximate to a location within the CT scanner, (ii) position information and orientation information of the physical object, with respect to the location, as determined from the 2D radiographs, and (iii) size information of the physical object determined from a predetermined distance between the location and the X-ray source. In some implementations, the computer data structure can include a 3D surface mesh.

In some implementations, the system can acquire (205) 2D radiographs of a physical object. In some implementations, the system can capture (210) images of the physical object using the at least one camera. In some implementations, the system can reconstruct (215) volumetric data for the physical object. In some implementations, the system can segment (220) the volumetric data. In some implementations, another system can perform one or more of the operations 205, 210, 215, and 220. In some implementations, the acquiring (205) and the capturing (210) can be performed concurrently.

The system adds (230) texture data to the 3D surfaces represented in the computer data structure using images captured by at least one optical camera. The images can be images of the physical object at different rotational orientations. In some implementations, the CT scanner can include a turntable. The location can be a center of the turntable. The different rotational orientations can correspond to different rotational positions of the turntable.

The system can add the texture data to the 3D surfaces by projecting data of the images onto the 3D surfaces using the size information, the position information, and the orientation information of the physical object and predetermined position, location, and parameters of the at least one optical camera. In some implementations, the system can project (232) the data of the images onto the 3D surfaces using a calibration transformation matrix. Details of generating the calibration transformation matrix are described herein in connection with FIGS. 4A-4C and FIG. 5.

In some implementations, the system can combine (234), at a single location on at least one of the 3D surfaces, data from at least two of the images of the physical object at the different rotational orientations. For example, for some locations on the 3D surface mesh, the system can combine data from multiple RGB images at different rotational positions of the turntable or from multiple cameras. The data from the multiple images can be averaged, summed, or combined in any suitable way to produce texture information for the location. The system provides (235) the computer data structure for processing of the 3D surfaces.

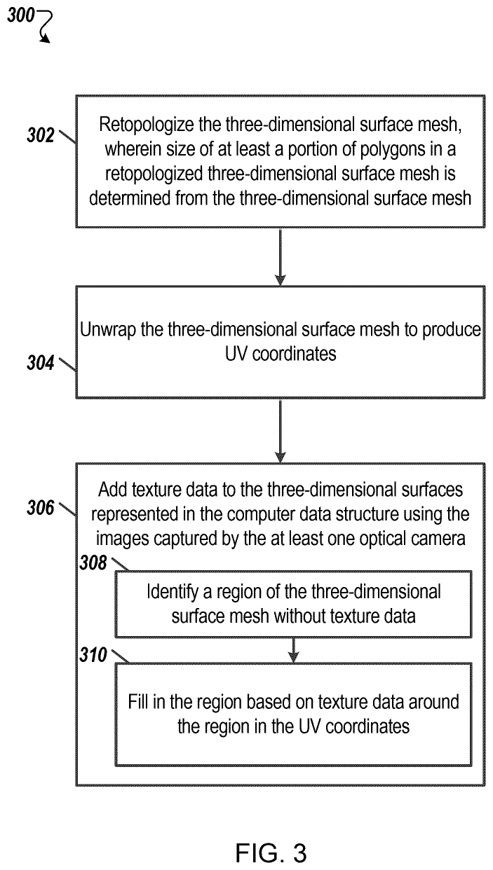

FIG. 3 is a flowchart showing an example of a process 300 to generate textured surfaces using UV coordinates. The process 300 can be performed by a system of one or more computers located in one or more locations. For example, a multimodal X-ray system, e.g., the system 100 depicted in FIG. 1, appropriately programmed in accordance with this specification, can perform the process 300.

In some implementations, the system can retopologize (302) the 3D surface mesh, and size of at least a portion of polygons in a retopologized 3D surface mesh can be determined from the 3D surface mesh. In some implementations, before projecting the data of the images onto a 3D surface mesh, the system can retopologize the 3D surface mesh. For each 3D surface mesh included in the computer data structure representing the 3D surfaces of the physical object, the system can retopologize the 3D surface mesh. In some implementations, the system can maintain the details of the 3D surfaces derived from the CT scan of the object by tuning the retopologization parameters. For example, the size of at least a portion of polygons in a retopologized 3D surface mesh can be determined from the 3D surface mesh that represents the details of the 3D surfaces derived from the CT scan. The system can tune the size of the polygons in the retopologizing process to preserve the desired level of surface detail. In some implementations, the system can perform non-uniform retopologizing. For example, the system can use smaller polygons in areas for either detected or user-indicated details, and can use larger polygons in areas where less details are required. In some implementations, the system can retopologize the 3D surface mesh based on curvature of the physical object represented in the 3D surface mesh, such using a curvature aware decimation method.

In some implementations, the system can retopologize the 3D surface mesh to produce optimized geometry that is suitable for use in surface texturing. In some implementations, when polygons of different layers of the 3D surface mesh occlude each other, the optimized geometry suitable for use in surface texturing does not include the polygons that are being occluded such that the optimized geometry after retopologizing can represent only the visible surface of an object. In some implementations, the system can generate this optimized geometry using a shrink wrap mesh generation algorithm. For example, if an object has multiple layers of material, the mesh that represents the surface of the object can omit the occluded layers because texture data is not available for the occluded layers that are not visible by the cameras.

The system can unwrap (304) the 3D surface mesh to produce UV coordinates used in the projecting. For example, the system can unwrap the mesh to produce a complimentary set of UV coordinates that prioritize adjacency and relative polygon scale. This set of UV coordinates can represent the coordinate translation between the texture coordinate space and the 3D surface mesh coordinate space.

The system can project the data of the images obtained from each camera onto the UV coordinates using the size information, position information, and orientation information of the physical object and predetermined position, location, and parameters of the at least one optical camera. For example, for each image, the system can determine the rotational position of the turntable. The system can determine a region of the 3D surface mesh corresponding to rotational position of the turntable using the predetermined position, location, and parameters of the camera that captured the image. The system can project the data of the image captured at the rotational position of the turntable onto the 3D surface mesh in the UV coordinate space, thus filling in some texture information in the UV coordinate space. Thus, for a given rotational position of the turntable, the system can add (306) some texture data to the 3D surfaces using the corresponding image captured at the given rotational position of the turntable.

In some implementations, the system can project the data of the images obtained from each camera onto the UV coordinates based on a calibration transformation matrix. The calibration transformation matrix projects an image of a scan object captured using the camera and in the two-dimensional coordinate system of the camera to the three-dimensional coordinate system of the computed tomography scanner. Details of generating the calibration transformation matrix are described herein in connection with FIGS. 4A-4C and FIG. 5. For example, the system can use the calibration transformation matrix to determine the relative locations between pixels in an image captured using a camera to a mesh extracted from a CT reconstruction. Based on the calibration transformation matrix, the system can overlay the texture on top of the mesh in precisely the right locations.

In some implementations, the system can identify (308) a region of the 3D surface mesh without texture data, and the system can fill (310) in the region based on texture data around the region in the UV coordinates. For example, the system can identify a region of the 3D surface without texture data or with limited texture data due to overhangs or occlusions, and the system can fill gaps in the texture data using texture data based on adjacency in the UV coordinate space.

To accurately add texture data to the 3D surfaces represented in the computer data structure using the images captured by the at least one optical camera, a multimodal X-ray system can calibrate location and orientation of the one or more cameras relative to the CT scanner. For example, to accurately project features from one or more images onto a 3D mesh, the system can specify the position and orientation (perspective) of the one or more optical cameras with respect to the X-ray source and detector of the CT scanner, and correct for the geometric distortions that those camera(s) introduce to the images that they capture.

FIGS. 4A-4C shows an example of a reference object 400 (referred to as a “phantom”) that can be used to calibrate location and orientation of one or more cameras relative to a CT scanner in a multimodal X-ray system that includes an X-ray scanner and one or more optical cameras, such as the system 100 in FIG. 1. The reference object 400 includes a plate 401 as the base of the reference object 400. The plate 401 has two opposite sides, such as the side 402 and the opposite side 404. The plate 401 can be held by a turntable of the CT scanner. For example, the plate 401 can be retained (e.g., using screws) by a fixture 108 that is attached to the turntable 105 of the CT scanner 110. For example, the plate 401 can be attached to an adapter that facilitates rigid attachment to the rotary sample stage, e.g., the turntable 105 of the CT scanner 110.

The reference object 400 includes a first set of features that can be detected in the X-ray domain using the X-ray scanner, and a second set of features that can be detected in the optical domain using the one or more cameras. The geometry within each set of features is controlled or measured. The geometry between the first set of features relative to the second set of features is also controlled or measured, i.e., known at the time of calibration of a specific CT scanner.

FIG. 4A shows an example of the side 402 of the reference object 400. On the side 402, the reference object 400 includes an X-ray detectable feature. The X-ray detectable feature can include three or more aspects. The three or more aspects can be at multiple fixed locations of the reference object 400. The three or more aspects can be fixed to the side 402 of the plate 401 using any appropriate methods, such as using an adhesive or using a threaded connection. For example, the reference object 400 can include alumina ceramic spheres adhered using epoxy. In some implementations, the X-ray detectable feature includes three or more spheres 406 fixed to the side 402 of the plate 401. In some implementations, the centers of the three or more aspects can be on the same 2D plane. For example, as shown in FIG. 4A, a set of twelve spheres can be affixed to one side of the plate 401 for detection and calibration in the X-ray domain. In some implementations, the centers of the three or more aspects are not required to be on the same 2D plane. Other shapes, different from spheres, can also be used for the X-ray detectable feature, such as cylinders or cubes. In some implementations, the X-ray detectable feature can include holes in a plate of known thickness. The X-ray detectable feature can occupy the FOV of the CT scanner, and can be evenly distributed or irregularly distributed on the side 402 of the plate 401.

The X-ray detectable feature can have a material that can be distinguished from a different material for the other parts of the phantom due to differences in X-ray attenuation of the materials. In some implementations, the absolute difference between an X-ray attenuation coefficient of the material for the X-ray detectable feature and an X-ray attenuation coefficient of the material for the plate 401 can be larger than a threshold. In some implementations, the absolute difference between the X-ray attenuation coefficient of the material for the X-ray detectable feature and the X-ray attenuation coefficient of the material for the plate 401 can be larger than the X-ray attenuation coefficient of the material for the plate 401 by a threshold percentage. For example, the X-ray attenuation coefficient of the material for the X-ray detectable feature can be at least 15%, 20%, or 25% greater than the X-ray attenuation coefficient of the material for the plate 401. In some implementations, the X-ray detectable feature can be made of metal or ceramic materials. For example, the X-ray detectable feature can be aluminum oxide spheres. The plate 401 can use plastic, acrylic, carbon fiber, PLA, or paper. In some cases, the optically detectable features (e.g., a sheet 408, which can be removable) can use acrylic, carbon fiber, PLA, paper, glass, or metal.

The size of the X-ray detectable feature can be selected to facilitate detection of the X-ray detectable feature in the reconstructed volume. For example, the diameter of the spheres can be selected to facilitate sphere detection in the reconstructed volume. For example, the diameter of the spheres can be within a range between 3 mm and 10 mm. The geometry within the set of three or more aspects included in the X-ray detectable feature can be controlled or measured. In some implementations, the center-to-center distance of each combination of sphere pairs can be predetermined. In some implementations, the geometry can be measured using a coordinate measuring system or other tools.

FIG. 4B shows an example of the opposite side 404 of the reference object 400. On the opposite side 404, the reference object 400 includes an optically detectable feature at a known (e.g., fixed) location of the reference object 400. The optically detectable feature can include a 2D pattern, e.g., on a piece of material that is attachable to the opposite side 404 of the plate 401 at a predetermined location. The 2D pattern can be placed on the opposite side 404 of the plate 401 to allow calibration and distortion correction in the optical domain. The 2D pattern can be a checkerboard pattern, dot grids, a ChArUco pattern, a Kalibr/ApriGrid pattern, or any other suitable fiducial markers arranged in a grid structure for camera or sensor calibration. In some implementations, the 2D pattern can be an arbitrary pattern that is detectable using a feature detector, such as a scale-invariant feature transform (SIFT) algorithm.

In some implementations, the optically detectable feature can include a sheet 408 of material having a 2D pattern, and the 2D pattern can have a predetermined size. For example, the optically detectable feature can include a sheet 408 of material having a checkerboard pattern 412 of 10 mm×10 mm squares. Other densities of the patterns are also possible, e.g., 2 mm×2 mm or 5 mm×5 mm. The 2D pattern can be produced by a variety of methods, such as inkjet printing, laser etching, or photolithography.

The relative location between the X-ray detectable feature and the optically detectable feature is predetermined. For example, the relative location between the three or more spheres 406 and the checkerboard pattern 412 can be predetermined. In some implementations, the X-ray detectable feature can be fixed to predetermined locations on the side 402 of the plate 401, and the sheet 408 of 2D patterns is attached to a predetermined location on the opposite side 404 of the plate 401. For example, two edges of the sheet 408 can be aligned with two corresponding edges of the plate 401.

In some implementations, the optically detectable feature can include a sheet 408 of material that is rigid and flat for better calibration accuracy of the one or more cameras. However, the sheet of material that is rigid and flat may have a higher attenuation coefficient than the attenuation coefficient of the plate 401. Therefore, if the sheet 408 is not removed, it may affect the detection of the X-ray detectable feature. For example, the sheet 408 of material for the 2D pattern can be glass or metal, e.g., a glass lithography checkerboard with high precision, but with relatively higher attenuation to the X-rays than other less rigid materials, such as paper or plastic.

In some implementations, the optically detectable feature and the X-ray detectable feature can be separable. The optically detectable feature can be removed from the plate 401 before the reference object is used to perform calibration of the X-ray scanner. FIG. 4C shows the opposite side 404 of the reference object after the optically detectable feature, e.g., the sheet 408 of the checkerboard pattern 412, has been removed. The optically detectable feature can be attached to the plate 401 at the fixed location before the reference object is used to perform calibration of the one or more optical cameras.

By decoupling the CT scanning and the camera imaging, the reference object 400 can use advantageous materials and sizes for the optically detectable feature. The optically detectable feature can use a flatter material with higher attenuation for improved precision of the calibration of the one or more cameras. Due to space constraint, a non-removable sheet can have a limited size to avoid collision with the interior of the enclosure 112 of the CT scanner 110 during rotation of the turntable 105 when capturing the 2D radiographs for calibrating the CT scanner 110. By decoupling the CT scanning and the camera imaging, the optically detectable feature can have a larger size, e.g., larger than the size of the plate 401, and therefore can be used to calibrate a larger field of view of the one or more cameras. For example, the optically detectable feature having a larger size can be used to calibrate a camera's intrinsic parameters, such as lens distortion, focal length, and principle point.

In some implementations, the optically detectable feature can occupy most of or all of the opposite side 404 of the plate 401, as shown in FIG. 4B. In some implementations, the optically detectable feature can occupy a portion of the opposite side 404 of the plate 401, not shown in FIG. 4B. For example, the system can calibrate a camera's intrinsic parameters outside of the CT scanner. After finishing the calibration of the camera's intrinsic parameters, the camera can be placed in the CT scanner. The system can use the optically detectable feature only for calculating the position and orientation of the camera with respect to the reference object (e.g., calibration of the camera's extrinsic parameters) and the optically detectable feature can only occupy a portion of the plate 401. For example, the sheet 408 can only cover half the area of the opposite side 404, thus providing half of the reference object 400 with a clear line of sight for X-rays to pass through the spheres 406.

In some implementations, the plate 401 can include an alignment feature 410. The alignment feature 410 can define a precise location of the optically detectable feature, e.g., the sheet 408 of the 2D pattern. For example, the plate 401 can be precisely machined or manufactured to include the alignment feature 410, e.g., edges defining a precise location of the left edge and the bottom edge of the sheet 408 of the checkerboard pattern 412. When installing the sheet, the sheet 408 can be placed right against the 410 for precise location of the optically detectable feature.

FIG. 5 is a flowchart showing an example of a process 500 of multimodal calibration using a reference object. The reference object can be the reference object described in FIGS. 4A-4C. The process 500 can be performed by a system of one or more computers located in one or more locations. For example, a multimodal X-ray system, e.g., the system 100 depicted in FIG. 1, appropriately programmed in accordance with this specification, can perform the process 500.

The system generates (502) data of an X-ray detectable feature represented in volumetric data reconstructed from 2D radiographs of a reference object acquired using a CT scanner. The reference object includes the X-ray detectable feature and an optically detectable feature. A relative location between the X-ray detectable feature and the optically detectable feature is predetermined.

For example, the system 100 can rotate a reference object, e.g., the reference object 400 in FIGS. 4A-4C, attached to the turntable 105. The reference object 400 can be the object 104 that is between the X-ray source 101 and the detector 103. The system 100 can acquire several 2D radiographs at different rotational orientations using the CT scanner 110. The system 100 can generate a reconstruction volume of the reference object from the 2D radiographs.

In some implementations, the X-ray detectable feature included in the reference object can be three or more spheres. The data of the X-ray detectable feature can include a point cloud of centers of the three or more spheres. For example, the system can generate a segmentation volume for the spheres from the reconstruction volume using a threshold. The system can determine the centers of the segmentations of the spheres. The system can generate a point cloud of the centers of the spheres.

In some implementations, the optically detectable feature can be removed from the reference object before the X-ray scanner acquires the 2D radiographs of the reference object. In some implementations, the optically detectable feature is not removed from the reference object while the X-ray scanner acquires the 2D radiographs of the reference object.

The system determines (504) a first transformation matrix using the data of the X-ray detectable feature in the volumetric data. The first transformation matrix aligns a 3D coordinate system of the CT scanner with a first volume space that defines the X-ray detectable feature. The first transformation matrix can project a point from the CT scanner coordinate system to a point in the X-ray volume space of the reconstructed X-ray detectable feature. For example, the first transformation matrix can be a 4×4 matrix that includes rotation and translation vectors that enable transforming a 3D point in the CT scanner coordinate system into a physical world coordinate of the X-ray volume space.

In some implementations, the X-ray detectable feature included in the reference object can be three or more aspects, such as three or more spheres, cylinders, or cubes. For example, at least three aspects can be used to determine a 4×4 first transformation matrix. The data of the X-ray detectable feature can include a point cloud of centers of the three or more aspects. The system can generate the first transformation matrix using a point cloud alignment algorithm that aligns the point cloud of the centers of the three or more aspects in the volumetric data with a point cloud of the centers of the three or more aspects in the 3D coordinate system of the X-ray scanner.

For example, the system can detect the spheres from the CT reconstruction volume and can determine a point cloud of the centers of the spheres in the CT reconstruction volume. The system can obtain a known point cloud of centers of the spheres in the reference object based on their known locations or from measurement of the placement of the spheres. The system can perform point cloud alignment with an alignment algorithm to find a transformation matrix that minimizes the error between the known point cloud of centers of the spheres in the reference object and the point cloud of the centers of the detected spheres in the reconstruction volume. The alignment algorithm can be any point cloud alignment/registration algorithm, such as Kabsch-Umeyama algorithm, or iterative closest point (ICP) algorithm.

As another example, the reference object can include three or more holes carved out from the plate, and the holes can have known sizes and shapes. The system can detect the holes from the CT reconstruction volume and can determine a point cloud of centers of the three or more holes in the CT reconstruction volume. The system can obtain a known point cloud of centers of the holes in the reference object based on their known locations or from measurement of the placement of the holes. The system can perform point cloud alignment with an alignment algorithm to find a transformation matrix that minimizes the error between the known point cloud of centers of the holes in the reference object and the point cloud of the centers of the detected holes in the reconstruction volume.

The system obtains (506) one or more reference images of the optically detectable feature included in the reference object. The one or more reference images are captured using each of one or more optical cameras. Each reference image is in a 2D coordinate system of the corresponding optical camera. In some implementations, the system can capture the reference image of the reference object. In some implementations, an end user of the system can perform the capturing of the reference image of the reference object and can provide the reference image to the system. For example, a server system in the cloud can receive the reference images transmitted over a network from a local computer at the X-ray scanner.

In some implementations, the optically detectable feature included in the reference object can include a 2D pattern on a surface associated with the reference object. In some implementations, the surface can be a sheet of material that is removable from the reference object. In some implementations, the system can capture one or more images of the optically detectable feature of the phantom using some or all cameras. In some implementations, the system can capture one or more images of the optically detectable feature of the phantom in one or more angular positions of the turntable. In some implementations, the system can perform the optical image acquisition (described in connection with operation 506) simultaneously with the radiograph acquisition (described in connection with operation 502). In some implementations, the system can perform the optical image acquisition (described in connection with operation 506) and the radiograph acquisition (described in connection with operation 502) sequentially. For example, the system can firstly perform the optical image acquisition, and can secondly perform the radiograph acquisition (e.g., after removing the optically detectable feature).

The system calculates (508), for each of the at least one optical camera, a second transformation matrix using the reference image of the optically detectable feature. The second transformation matrix aligns a second volume space that defines the optically detectable feature with the 2D coordinate system of the optical camera. The second transformation matrix can project a point from the 3D coordinate system of the reference object as defined by the optically detectable feature to a point in the 2D coordinate system of the optical camera. For example, the second transformation matrix can be a 4×4 matrix that includes rotation and translation vectors that allow transforming a point in the 2D coordinate system of the optical camera into a physical world coordinate of the X-ray volume space.

In some implementations, the system can first perform geometric distortions correction introduced by the cameras using ground-truth knowledge of the optically detectable feature before calculating the second transformation matrix. For example, using ground-truth knowledge of the checkerboard pattern in the reference object, the system can (i) correct for image distortions (e.g., calibrating the camera's intrinsic parameters), and (ii) calculate the position and orientation of each camera with respect to the reference object (e.g., calibrating camera's extrinsic parameters).

In some implementations, the system can calibrate the camera's intrinsic parameters by capturing multiple images of the optically detectable feature at different positions and angles. The system can find lens model(s) and distortion parameters. After this calibration is performed, the system can generate images that are calibrated where distances are actually representative of distances in the physical world.

In some implementations, the system can calibrate the camera's extrinsic parameters, e.g., determining the second transformation matrix, by finding the pose of the 2D pattern included in the optically detectable feature relative to the camera (and vice versa) in real world coordinates. The second transformation matrix can be a 4×4 matrix that includes rotation and translation that transforms points from a world coordinate (e.g., the second volume space that defines the optically detectable feature) to the camera coordinate system.

The system produces (510), for each of the at least one optical camera, a calibration transformation matrix using (i) the first transformation matrix, (ii) the second transformation matrix, and (iii) a third transformation matrix. The third transformation matrix aligns the first volume space and the second volume space based on the predetermined relative location between the X-ray detectable feature and the optically detectable feature in the reference object. In some implementations, the third transformation matrix can characterize the translation and/or rotation offset between the X-ray detectable feature and the optically detectable feature. For example, the transformation matrix can be a 4×4 matrix characterizing the offset between the spheres and the sheet of the checkerboard pattern with rotation component, translation component, or both. The calibration transformation matrix projects an image of a scan object captured using the optical camera and in the 2D coordinate system of the optical camera to the 3D coordinate system of the CT scanner.

For example, the system can compare the location and position information determined and calculated in operations 504 and 508, along with knowledge of the geometry offset (e.g., using the third transformation matrix) between X-ray detectable feature and the optically detectable feature, to locate and orient each of the one or more optical cameras with respect to the X-ray source and detector of the CT scanner. The system can generate a 4×4 calibration transformation matrix that projects points from the CT coordinate system to the camera coordinate system of each camera, or vice versa.

In some implementations, the process 500 can be repeated in several positions of the reference object 400 to improve the accuracy of the calibration of the positions and orientations of the one or more cameras relative to the CT scanner. In some implementations, the CT scanner can have a moveable gantry that is previously calibrated relative to the detector and X-ray source of the CT scanner. The system can perform the calibration of the positions and orientations of the one or more cameras relative to the CT scanner in arbitrary positions. The system can apply a gantry transformation that follows from a move from the calibrated position A to an arbitrary position B for the reference object 400. For example, a properly calibrated CT system can obtain a position of the reference object relative to a predetermined origin. When moving the reference object in the CT scanner with a known distance in 3D (e.g., distances in X, Y, Z directions), the system can calculate the 3D location of the reconstruction volume of the reference object. The system can perform the camera calibration when the reference object is placed in any location in the CT scanner because the system can obtain the relative location between the camera and the reference object. The system can calculate the calibration transformation matrix based on the known transformation from the reference object's location in the coordinate system of the X-ray scanner to the coordinate system of the camera.

The system provides (512) the calibration transformation matrix, which is used in aligning an image of a physical object obtained using the optical camera with a CT reconstruction volume of the physical object obtained using the X-ray scanner. In some implementations, a server system in the cloud can receive the reference images transmitted over a network from a local computer at the X-ray scanner, determine a calibration transformation matrix, and send back the calibration transformation matrix to the local computer. The local computer can calibrate location and orientation of the one or more cameras relative to the CT scanner using the calibration transformation matrix. In some implementations, the same system can produce the calibration transformation matrix and can use the calibration transformation matrix for aligning an image of a physical object obtained using the optical camera with a CT reconstruction volume of the physical object obtained using the X-ray scanner.

Using the calibration transformation matrix, the system can project features from images captured by each camera onto a CT reconstruction volume of an object scanned using the CT scanner. In some implementations, the system can obtain a computer data structure (e.g., a 3D surface mesh) representing 3D surfaces of a physical object from a volumetric data reconstructed from 2D radiographs of the physical object acquired using the X-ray scanner. The system can add texture data to the 3D surfaces represented in the computer data structure using images captured by the one or more optical cameras. The images can be images of the physical object at different rotational orientations. The system can add the texture data to the 3D surfaces by projecting data of the images onto the 3D surfaces using the calibration transformation matrix. Details of generating the textured surfaces using a multimodal X-ray system are described herein in connection with FIGS. 1-3.

In a reconstruction volume generated from a CT scan, a boundary of an object can be blurry. Some techniques can determine a boundary of an object using a threshold value that is determined based on the range of values for the blurry boundary. For example, according to the ISO-50 method, if the range for a blurry boundary is from 0 to 100, some systems can determine to use a threshold value at 50% of the range, e.g., a threshold value at 50 as the cutoff for the boundary. However, these techniques may not generate data for the boundary that corresponds to the true boundary of the object.

A multimodal X-ray imaging system, such as the system 100, can accurately determine a boundary of an object by comparing one or more images of the object to a reconstruction volume generated from a CT scan. FIG. 6 is a flowchart showing an example of a process 600 to determine an object boundary in a reconstruction volume using a camera image. The process 600 can be performed by a system of one or more computers located in one or more locations. For example, a multimodal X-ray system, e.g., the system 100 depicted in FIG. 1, appropriately programmed in accordance with this specification, can perform the process 600.

The system can obtain (602) volumetric data reconstructed from 2D radiographs of a physical object acquired using the X-ray scanner. The system can generate (604) projected data by projecting data of images captured by the one or more optical cameras to a 3D coordinate system of the volumetric data using the calibration transformation matrix. The images can be images of the physical object at different rotational orientations.

The system can determine (606) 3D surfaces (e.g., a boundary) of the physical object from the volumetric data using a boundary of the physical object represented in the projected data of the images of the physical object. In some implementations, the system can determine a threshold for segmenting the 3D surfaces based on the images of the physical object captured by the one or more optical cameras.

In some implementations, after determining the 3D surfaces, the system 100 can extract a surface mesh from the volumetric data, e.g., a CT reconstruction volume, based on the 3D surfaces. In some implementations, the system can generate accurate surface mesh of the physical object, as described in operation 225 of FIG. 2. In some implementations, the surface mesh can be provided for a subsequent processing, such as adding texture data to the surface mesh, as described in operation 230 of FIG. 2.

When performing a CT scan of an object (e.g., a manufactured part), it is desirable to know the extent of an object, e.g., a 3D space that the object occupies when the object rotates within a CT scanner. In some cases, it is important to make sure that the object is within a FOV of a CT scanner, including while the object rotates on a turntable, such that the entire object can be fully scanned and reconstructed. To make sure the object is within the FOV, some systems require repeatedly scanning and repositing an object until the object is perfectly centered in the CT scanner. In some cases, an asymmetric object that is not centered on the turntable may be outside the FOV of the CT scanner. In some cases, a motion system of a CT scanner can move the object out of the FOV of the CT scanner before taking X-ray radiographs that are used to correct for detector imperfections, such as for flat field correction. Without knowing the extent of the object, the object may crash into the interior of the enclosure of the CT scanner.

FIG. 7 is a flowchart showing an example of a process 700 to estimate part occupation. The process 700 can be performed by a system of one or more computers located in one or more locations. For example, a multimodal X-ray system, e.g., the system 100 depicted in FIG. 1, appropriately programmed in accordance with this specification, can perform the process 700.

The system can detect (702) boundaries of a physical object in images captured by the one or more optical cameras. The images can be images of the physical object at different rotational orientations. The system can project (704), using the calibration transformation matrix, data of the boundaries of the physical object in the images to a 3D coordinate system of the X-ray scanner to generate a 3D boundary of the physical object in the X-ray scanner. The system can predict (706), using the 3D boundary of the physical object in the X-ray scanner, a 3D space that the physical object occupies when the physical object moves within the X-ray scanner. For example, the system can generate a surface mesh with photogrammetry techniques and can use the surface mesh to determine the location and the dimensions of the physical object.

In some implementations, the system can calculate (708) the 3D space that the physical object occupies when the physical object is moved outside a field-of-view of the X-ray scanner. For example, when performing correction for detector imperfections, such as flat field correction, the system may move a part out of the FOV of the CT scanner in order to take radiographs that have no part in the view. Based on the calculated 3D space that the physical object occupies, the system can determine the extent of the part relative to the interior of the enclosure of the CT scanner. The system can predict whether the part would hit the interior of the enclosure of the CT scanner when the part was to be moved out of the FOV of the CT scanner. If the system determines that the part would not hit the interior of the enclosure of the CT scanner, the system can proceed to move the part out of the FOV for performing the correction for detector imperfections. If the system determines that the part would hit the interior of the enclosure of the CT scanner, the system can determine to not move the part out of the FOV, or the system can determine to only move the part partially out of the FOV, to avoid any potential collision.

In some implementations, the X-ray scanner can include a turntable, and the physical object can be placed on or attached to the turntable. The system can calculate (710) the 3D space that the physical object occupies when the physical object is rotated to different rotational positions of the turntable. Instead of repeatedly scanning and repositing a physical object until the physical object is perfectly centered in the CT scanner, the system can determine the 3D space that the physical object occupies based on the images captured by the one or more optical camera.

In some implementations, the system can determine (712) a target location for the physical object in the X-ray scanner based on the predicted 3D space that the physical object occupies when the physical object moves within the X-ray scanner. In some implementations, the system can move (714) the physical object to the target location in the X-ray scanner. In some implementations, based on the 3D space that the physical object occupies, the system can determine the distance for a repositioning that can center the physical object in the CT scanner, or can ensure that the physical object is within the FOV of the CT scanner. For example, based on the 3D space that the physical object occupies, the system can determine to move the object by a distance value, e.g., 2 mm, to the left such that the physical object can be properly centered in the CT scanner.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented using one or more modules of computer program instructions encoded on a non-transitory computer-readable medium for execution by, or to control the operation of, data processing apparatus. The computer-readable medium can be a manufactured product, such as a hard drive in a computer system or an optical disc sold through retail channels, or an embedded system. The computer-readable medium can be acquired separately and later encoded with the one or more modules of computer program instructions, such as by delivery of the one or more modules of computer program instructions over a wired or wireless network. The computer-readable medium can be a machine-readable storage device, a machine-readable storage substrate, a memory device, or a combination of one or more of them.

The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, a runtime environment, or a combination of one or more of them. In addition, the apparatus can employ various different computing model infrastructures, such as web services, distributed computing, and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media, and memory devices, including by way of example semiconductor memory devices, e.g., EPROM (Erasable Programmable Read-Only Memory), EEPROM (Electrically Erasable Programmable Read-Only Memory), and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., an LCD (liquid crystal display) display device, an OLED (organic light emitting diode) display device, or another monitor, for displaying information to the user, and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The computing system can include clients and servers. A client and server are generally remote from each other and typically interact through a communication network. The relationship of client and server arises by virtue of computer programs running on the respective computers and having a client-server relationship to each other. Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described is this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While this specification contains many implementation details, these should not be construed as limitations on the scope of what is being or may be claimed, but rather as descriptions of features specific to particular embodiments of the disclosed subject matter.

Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desired results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the invention have been described. Other embodiments are within the scope of the following claims.

### Examples

Although the present application is defined in the attached claims, it should be understood that the present invention can also (additionally or alternatively) be defined in accordance with the following examples:

Example 1: A method comprising:
generating a computer data structure representing three-dimensional surfaces of a physical object from (i) segmentation of volumetric data reconstructed from two-dimensional radiographs of the physical object acquired using an X-ray source in a computed tomography scanner when the physical object is proximate to a location within the computed tomography scanner, (ii) position information and orientation information of the physical object, with respect to the location, as determined from the two-dimensional radiographs, and (iii) size information of the physical object determined from a predetermined distance between the location and the X-ray source;
adding texture data to the three-dimensional surfaces represented in the computer data structure using images captured by at least one optical camera, the images being of the physical object at different rotational orientations, by projecting data of the images onto the three-dimensional surfaces using the size information, the position information, and the orientation information of the physical object and predetermined position, location, and parameters of the at least one optical camera; and
providing the computer data structure for processing of the three-dimensional surfaces.

Example 2: The method of Example 1, wherein the adding comprises combining, at a single location on at least one of the three-dimensional surfaces, data from at least two of the images of the physical object at the different rotational orientations.

Example 3: The method of any one of the previous Examples, wherein the computer data structure comprises a three-dimensional surface mesh.

Example 4: The method of any one of the previous Examples, comprising:
retopologizing the three-dimensional surface mesh, wherein size of at least a portion of polygons in a retopologized three-dimensional surface mesh is determined from the three-dimensional surface mesh; and
unwrapping the three-dimensional surface mesh to produce UV coordinates used in the projecting.

Example 5: The method of any one of the previous Examples, wherein the adding comprises:
identifying a region of the three-dimensional surface mesh without texture data; and
filling in the region based on texture data around the region in the UV coordinates.

Example 6: The method of any one of the previous Examples, comprising:
acquiring the two-dimensional radiographs;
reconstructing the volumetric data;
segmenting the volumetric data; and
capturing the images.

Example 7: The method of any one of the previous Examples, wherein the acquiring and the capturing are performed concurrently.

Example 8: The method of any one of the previous Examples, wherein the computed tomography scanner comprises a turntable, the location is a center of the turntable, and the different rotational orientations correspond to different rotational positions of the turntable.

Example 9: The method of any one of the previous Examples, comprising:
generating data of an X-ray detectable feature represented in volumetric data reconstructed from two-dimensional radiographs of a reference object acquired using the computed tomography scanner, wherein the reference object comprises the X-ray detectable feature and an optically detectable feature, wherein a relative location between the X-ray detectable feature and the optically detectable feature is predetermined;
determining a first transformation matrix using the data of the X-ray detectable feature in the volumetric data, wherein the first transformation matrix aligns a three-dimensional coordinate system of the computed tomography scanner with a first volume space that defines the X-ray detectable feature;
obtaining one or more reference images of the optically detectable feature included in the reference object, wherein the one or more reference images are captured using each of one or more optical cameras and each reference image is in a two-dimensional coordinate system of the corresponding optical camera;
calculating, for each of the at least one optical camera, a second transformation matrix using the reference image of the optically detectable feature, wherein the second transformation matrix aligns a second volume space that defines the optically detectable feature with the two-dimensional coordinate system of the optical camera; and
producing, for each of the at least one optical camera, a calibration transformation matrix using (i) the first transformation matrix, (ii) the second transformation matrix, and (iii) a third transformation matrix that aligns the first volume space and the second volume space based on the predetermined relative location between the X-ray detectable feature and the optically detectable feature in the reference object, wherein the calibration transformation matrix projects an image of a scan object captured using the optical camera and in the two-dimensional coordinate system of the optical camera to the three-dimensional coordinate system of the computed tomography scanner.

Example 10: The method of any one of the previous Examples, wherein the adding comprises adding the texture data to the three-dimensional surfaces represented in the computer data structure using the images captured by the at least one optical camera by projecting the data of the images onto the three-dimensional surfaces using the calibration transformation matrix.

Similar operations and processes as described in Examples 1 to 10 can be performed in a system comprising a data processing apparatus including at least one hardware processor and a non-transitory computer-readable medium encoding instructions configured to cause the data processing apparatus to perform the operations. Further, a non-transitory computer-readable medium encoding instructions operable to cause data processing apparatus to perform the operations as describes in any one of the Examples 1 to 10 can also be implemented.

What is claimed is:

1. A method comprising:
generating a computer data structure representing three-dimensional surfaces of a physical object from (i) segmentation of volumetric data reconstructed from two-dimensional radiographs of the physical object acquired using an X-ray source in a computed tomography scanner when the physical object is proximate to a location within the computed tomography scanner, (ii) position information and orientation information of the physical object, with respect to the location, as determined from the two-dimensional radiographs, and (iii) size information of the physical object determined from a predetermined distance between the location and the X-ray source;
adding texture data to the three-dimensional surfaces represented in the computer data structure using images captured by at least one optical camera, the images being of the physical object at different rotational orientations, by projecting data of the images onto the three-dimensional surfaces using the size information, the position information, and the orientation information of the physical object and predetermined position, location, and parameters of the at least one optical camera; and
providing the computer data structure for processing of the three-dimensional surfaces.

2. The method of claim 1, wherein the adding comprises combining, at a single location on at least one of the three-dimensional surfaces, data from at least two of the images of the physical object at the different rotational orientations.

3. The method of claim 1, wherein the computer data structure comprises a three-dimensional surface mesh.

4. The method of claim 3, comprising:
retopologizing the three-dimensional surface mesh, wherein size of at least a portion of polygons in a retopologized three-dimensional surface mesh is determined from the three-dimensional surface mesh; and
unwrapping the three-dimensional surface mesh to produce UV coordinates used in the projecting.

5. The method of claim 4, wherein the adding comprises:
identifying a region of the three-dimensional surface mesh without texture data; and
filling in the region based on texture data around the region in the UV coordinates.

6. The method of claim 1, comprising:
acquiring the two-dimensional radiographs;
reconstructing the volumetric data;
segmenting the volumetric data; and
capturing the images.

7. The method of claim 6, wherein the acquiring and the capturing are performed concurrently.

8. The method of claim 1, wherein the computed tomography scanner comprises a turntable, the location is a center of the turntable, and the different rotational orientations correspond to different rotational positions of the turntable.

9. The method of claim 1, comprising:
generating data of an X-ray detectable feature represented in volumetric data reconstructed from two-dimensional radiographs of a reference object acquired using the computed tomography scanner, wherein the reference object comprises the X-ray detectable feature and an optically detectable feature, wherein a relative location between the X-ray detectable feature and the optically detectable feature is predetermined;
determining a first transformation matrix using the data of the X-ray detectable feature in the volumetric data, wherein the first transformation matrix aligns a three-dimensional coordinate system of the computed tomography scanner with a first volume space that defines the X-ray detectable feature;
obtaining one or more reference images of the optically detectable feature included in the reference object, wherein the one or more reference images are captured using each of one or more optical cameras and each reference image is in a two-dimensional coordinate system of the corresponding optical camera;
calculating, for each of the at least one optical camera, a second transformation matrix using the reference image of the optically detectable feature, wherein the second transformation matrix aligns a second volume space that defines the optically detectable feature with the two-dimensional coordinate system of the optical camera; and
producing, for each of the at least one optical camera, a calibration transformation matrix using (i) the first transformation matrix, (ii) the second transformation matrix, and (iii) a third transformation matrix that aligns the first volume space and the second volume space based on the predetermined relative location between the X-ray detectable feature and the optically detectable feature in the reference object, wherein the calibration transformation matrix projects an image of a scan object captured using the optical camera and in the two-dimensional coordinate system of the optical camera to the three-dimensional coordinate system of the computed tomography scanner.

10. The method of claim 9, wherein the adding comprises adding the texture data to the three-dimensional surfaces represented in the computer data structure using the images captured by the at least one optical camera by projecting the data of the images onto the three-dimensional surfaces using the calibration transformation matrix.

11. A system comprising:
a data processing apparatus including at least one hardware processor; and
a non-transitory computer-readable medium encoding instructions configured to cause the data processing apparatus to perform operations comprising:
generating a computer data structure representing three-dimensional surfaces of a physical object from (i) segmentation of volumetric data reconstructed from two-dimensional radiographs of the physical object acquired using an X-ray source in a computed tomography scanner when the physical object is proximate to a location within the computed tomography scanner, (ii) position information and orientation information of the physical object, with respect to the location, as determined from the two-dimensional radiographs, and (iii) size information of the physical object determined from a predetermined distance between the location and the X-ray source;
adding texture data to the three-dimensional surfaces represented in the computer data structure using images captured by at least one optical camera, the images being of the physical object at different rotational orientations, by projecting data of the images onto the three-dimensional surfaces using the size information, the position information, and the orientation information of the physical object and predetermined position, location, and parameters of the at least one optical camera; and
providing the computer data structure for processing of the three-dimensional surfaces.

12. The system of claim 11, wherein the adding comprises combining, at a single location on at least one of the three-dimensional surfaces, data from at least two of the images of the physical object at the different rotational orientations.

13. The system of claim 11, wherein the computer data structure comprises a three-dimensional surface mesh.

14. The system of claim 13, wherein the operations comprise:
retopologizing the three-dimensional surface mesh, wherein size of at least a portion of polygons in a retopologized three-dimensional surface mesh is determined from the three-dimensional surface mesh; and
unwrapping the three-dimensional surface mesh to produce UV coordinates used in the projecting.

15. The system of claim 14, wherein the adding comprises:
identifying a region of the three-dimensional surface mesh without texture data; and
filling in the region based on texture data around the region in the UV coordinates.

16. The system of claim 11, wherein the operations comprise:
acquiring the two-dimensional radiographs;
reconstructing the volumetric data;
segmenting the volumetric data; and
capturing the images.

17. The system of claim 16, wherein the acquiring and the capturing are performed concurrently.

18. The system of claim 11, wherein the computed tomography scanner comprises a turntable, the location is a center of the turntable, and the different rotational orientations correspond to different rotational positions of the turntable.

19. The system of claim 11, wherein the operations comprise:
generating data of an X-ray detectable feature represented in volumetric data reconstructed from two-dimensional radiographs of a reference object acquired using the computed tomography scanner, wherein the reference object comprises the X-ray detectable feature and an optically detectable feature, wherein a relative location between the X-ray detectable feature and the optically detectable feature is predetermined;
determining a first transformation matrix using the data of the X-ray detectable feature in the volumetric data, wherein the first transformation matrix aligns a three-dimensional coordinate system of the computed tomography scanner with a first volume space that defines the X-ray detectable feature;
obtaining one or more reference images of the optically detectable feature included in the reference object, wherein the one or more reference images are captured using each of one or more optical cameras and each reference image is in a two-dimensional coordinate system of the corresponding optical camera;
calculating, for each of the at least one optical camera, a second transformation matrix using the reference image of the optically detectable feature, wherein the second transformation matrix aligns a second volume space that defines the optically detectable feature with the two-dimensional coordinate system of the optical camera; and
producing, for each of the at least one optical camera, a calibration transformation matrix using (i) the first transformation matrix, (ii) the second transformation matrix, and (iii) a third transformation matrix that aligns the first volume space and the second volume space based on the predetermined relative location between the X-ray detectable feature and the optically detectable feature in the reference object, wherein the calibration transformation matrix projects an image of a scan object captured using the optical camera and in the two-dimensional coordinate system of the optical camera to the three-dimensional coordinate system of the computed tomography scanner.

20. The system of claim 19, wherein the adding comprises adding the texture data to the three-dimensional surfaces represented in the computer data structure using the images captured by the at least one optical camera by projecting the data of the images onto the three-dimensional surfaces using the calibration transformation matrix.

21. A non-transitory computer-readable medium encoding instructions operable to cause data processing apparatus to perform operations comprising:
generating a computer data structure representing three-dimensional surfaces of a physical object from (i) segmentation of volumetric data reconstructed from two-dimensional radiographs of the physical object acquired using an X-ray source in a computed tomography scanner when the physical object is proximate to a location within the computed tomography scanner, (ii) position information and orientation information of the physical object, with respect to the location, as determined from the two-dimensional radiographs, and (iii) size information of the physical object determined from a predetermined distance between the location and the X-ray source;
adding texture data to the three-dimensional surfaces represented in the computer data structure using images captured by at least one optical camera, the images being of the physical object at different rotational orientations, by projecting data of the images onto the three-dimensional surfaces using the size information, the position information, and the orientation information of the physical object and predetermined position, location, and parameters of the at least one optical camera; and
providing the computer data structure for processing of the three-dimensional surfaces.
